(19) United States
(12) Patent Application Publication
(10) Pub. No.: US 2005/0101953 A1
(43) Pub. Date: May 12, 2005

(54) Artificial facet joint and method

(76) Inventor: Peter M. Simonson, Miami Beach, FL (US)

Correspondence Address: AKERMAN SENTERFITT, P.O. BOX 3188, WEST PALM BEACH, FL, 33402-3188, US

(21) Appl. No.: 10/704,868

(22) Filed: Nov. 10, 2003

(51) Int. Cl. A61B 17/56; A61B 17/58

(52) U.S. Cl. 606/61

(57) ABSTRACT

An artificial facet joint includes a pair of connectors, each having a first device connecting member having structure for slidably engaging a rod, and a second device connecting member having an aperture for slidably engaging a screw. The first and second device connecting members are rotatably engaged to one another. A spinal implant and spinal implant screws are also provided. The first device connecting member of each connector is slidably engaged to the rod and the second device connecting member of each connector is slidably engaged to a respective one of the pair of spinal implant screws, whereby the screws can be engaged to the pedicles on one lateral side of adjacent vertebrae and the rod and connectors will limit movement of the joint. Another artificial facet joint can be provided on another lateral side of the adjacent vertebrae. A transverse member can be connected between the rods on each lateral side of the vertebrae.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

This invention relates generally to the field of artificial joints and more particularly to artificial joints and ligaments.

### BACKGROUND OF THE INVENTION

Each vertebra in the human spine has two sets of joints which interact with adjacent upper and lower joints. These joints are known as the facet joints, and are otherwise known as the zygapophyseal or apophyseal joints. Two joints are formed on each lateral side of the vertebra. The superior articular facet faces upward and the inferior articular facet faces downward, such that the superior articular facet of a lower vertebrae abuts the inferior articular facet of an adjacent upper vertebrae. The facet joints are located on the posterior of the spine adjacent the pedicle, lamina, and transverse process. The facet joints generally are hinge-like and allow limited flexion, extension, and twisting motion, while preventing excessive motion which could damage the spinal chord.

Various spinal reconstructive or treatment procedures require the removal of the facet joint and ligament structures. The joint and ligament must then be reconstructed artificially. Known artificial facet joints fail to provide the rigidity that is necessary to support the spine while permitting the flexibility to reassemble the facet joint.

### SUMMARY OF THE INVENTION

An artificial facet joint includes a pair of connectors. Each connector comprises a first device connecting member having structure for sliding engagement of a rod and a second device connecting member having structure for sliding engagement of a screw. The first device connecting member and second device connecting member are rotatably engaged to one another. A spinal implant rod and a pair of spinal implant screws are provided. The first device connecting member of each of the connectors is slidably engaged to the rod. The second device connecting member of each of the connectors is slidably engaged to a respective one of the pair of spinal implant screws. The screws can be engaged to the pedicles on one lateral side of adjacent vertebra and the rods and connectors will limit movement of the joint. Structure for securing the spinal implant rod against axial movement relative to the spine can be provided. The structure for sliding engagement of the rod can be an aperture and the structure for sliding engagement of a screw can be an aperture. The apertures of the first device connecting member and the second device connecting member can comprise a reduced friction coating.

The artificial facet joint can further comprise a second pair of connectors, a second spinal implant rod and a second pair of second implant screws. The second pair of screws can be engaged to the pedicles of the other lateral side of the adjacent vertebra and the second rod and second pair of connectors will limit movement of the joint on the other lateral side of the adjacent vertebra. A transverse member can be connected between the first and second rod. The transverse member can be slidably engaged to the first and second rods. The transverse member can alternatively be connected between screws.

The artificial facet joint can further comprise structure for securing the rod to a portion of the spine. This structure can comprise a clamp for the rod and structure for securing the clamp to a screw. Alternatively, the structure can comprise blocking members on the rod.

The spinal implant rod can include structure for engaging the first device connecting member so as to limit the sliding movement of the rod relative to the first device connecting member. The spinal implant screw can comprise structure for engaging the second device connecting member so as to limit the sliding movement of the rod relative to the second device connecting member.

A connector for an artificial facet joint includes a first device connecting member having structure for sliding engagement of a spinal implant rod and a second device connecting member having structure for sliding engagement of a spinal implant screw. The first device connecting member and second device connecting member are rotatably engaged to one another. The structure for engaging the first device connecting member can be an aperture and the structure for engaging the second device connecting member can be an aperture. The apertures of the first device connecting member and the second device connecting member can comprise a reduced friction coating.

A connector assembly for an artificial joint can include a connection device having a first connecting portion with structure for sliding engagement of a rod and a second connecting portion with sliding engagement of a screw. A spinal implant rod is slidably engaged to the first connecting portion and the spinal implant screw is slidably engaged to the second connecting portion. The structure for engaging the rod can be an aperture and the structure for engaging the screw can be an aperture. The spinal implant rod can comprise structure for engaging the first connecting portion so as to limit the sliding movement of the rod relative to the first connecting portion. The spinal implant screw can comprise structure for engaging the second connecting portion so as to limit the sliding movement of the rod relative to the second connecting portion.

An artificial facet joint includes a spinal implant rod and connector. The connector comprises a first device connecting member having structure for sliding engagement of said rod and a second device connecting member having structure for sliding engagement of a screw. The first device connecting member and second device connecting member are rotatably engaged to one another. Structure is provided for securing the spinal implant rod against axial movement relative to the spine.

A method for creating an artificial facet joint includes the step of providing a first pair of connectors. Each connector comprises a rod connecting member having an aperture for engaging a rod, screw connecting member having an aperture for engaging a screw, the rod connecting member and the screw connecting member being rotatably engaged to one another. A first screw is secured to a pedicle of a first vertebra. A second screw is secured to a pedicle of a second vertebra. The screws can be positioned in the plane of the facet. The screw connecting member of the first connector is slidably engaged to the first screw, and the screw connecting member of the second connector is slidably engaged to the second screw. A spinal implant rod is slidably engaged to the rod connecting member of the first connector and to the rod connecting member of the second connector. The rod is then secured.

A second pair of connectors can be provided. Each connector comprises a rod connecting member having an aperture for engaging a rod and a screw connecting member having an aperture for engaging a screw. The rod connecting member and the screw connecting member are rotatably engaged to one another. A first screw is secured to a pedicle on an opposite lateral side of a first vertebra. A second screw is secured to a pedicle on an opposite side of a second vertebra. The screw connecting member of the first connector is slidably engaged to the first screw and the screw connecting member of the second connector is slidably engaged to the second screw. A spinal implant rod is slidably engaged to the rod connecting member of the first connector of the second pair of connectors and to the rod connecting member of the second connector on the opposite lateral side of the vertebra. The second rod is secured between the second pair of connectors. A transverse member can be attached between the spinal implant rods.

A spinal joint assembly includes a spinal joint device joined to a spinal implant rod which is capable of post-operative sliding movement relative to the rod. Structure can be provided for limiting the length of sliding movement between the spinal implant rod and the spinal joint device. A method of connecting a spinal joint assembly to a spine includes the steps of connecting a spinal implant rod to a spine and attaching a spinal implant device to the rod. The device is capable of post-operative sliding movement relative to the rod.

A spinal joint assembly comprises a spinal joint device joined to a spinal implant screw. The spinal joint device is capable of post-operative sliding movement relative to the screw. Structure can be provided for limiting the length of sliding movement between the spinal implant screw and the spinal joint device. A method of connecting a spinal joint assembly to a spine includes the steps of connecting a spinal implant screw having a long axis to the spine. A spinal implant device is connected to the screw and is capable of post-operative sliding movement along the long axis of the screw.

A bone implant screw is provided for securing connected implants to a spine. The bone implant screw upon installation in the spine permits dorsal movement relative to itself and the connected implants. The screw can comprise a post. The movement permitted by the screw can further comprise rotation of the connected implants about an axis of the screw. The screw can comprise structure for limiting dorsal movement of the connected implants beyond a range of movement.

### BRIEF DESCRIPTION OF THE DRAWINGS

There are shown in the drawings embodiments which are presently preferred, it being understood, however, that the invention is not limited to the precise arrangements and instrumentalities shown, wherein:

FIG. 1 is a side elevation of a connector.

FIG. 2 is a plan view.

FIG. 3 is a perspective view of a connection assembly with a connector, spinal implant rod, and a spinal implant screw, illustrating by arrows the motion that is possible.

FIG. 4 is a perspective view of an artificial facet joint according to the invention as implanted in a spine.

FIG. 5 is a posterior view.

### DETAILED DESCRIPTION OF THE INVENTION

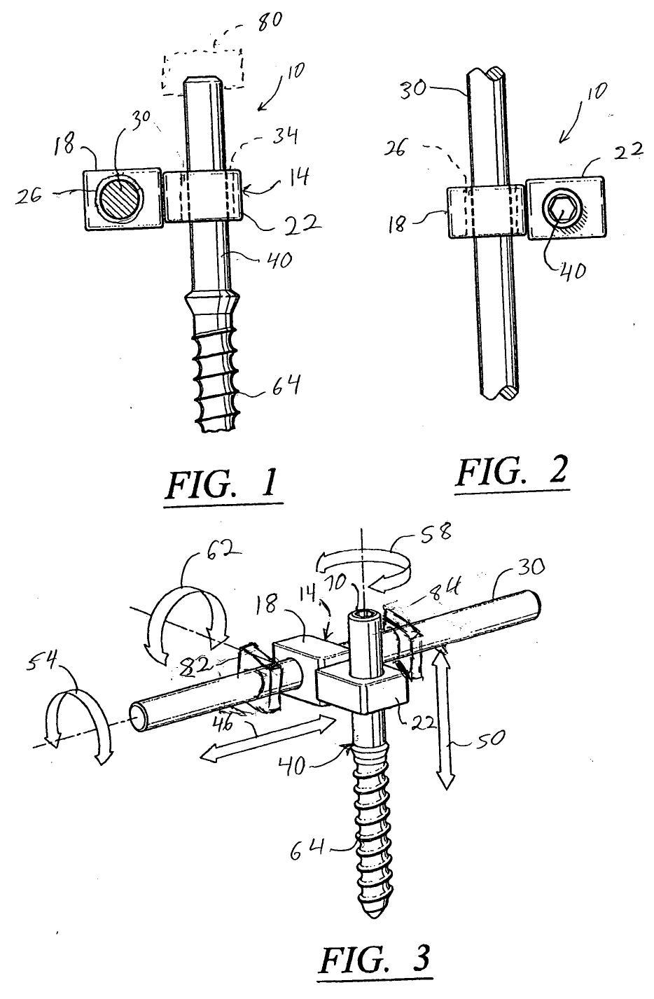

There is shown in FIGS. 1-3 a connector assembly for an artificial facet joint according to the invention. The connector assembly 10 includes a connector 14 having a first device connecting member 18 and a second device connecting member 22. The first device connecting member 18 has structure for sliding engagement of a spinal implant rod 30. The second device connecting member 22 has structure for sliding engagement of a spinal implant screw 40. The structure for slidably engaging the spinal implant rod 30 can be an aperture 26 for receiving the rod 30. The structure for slidably engaging the spinal implant screw 40 can be an aperture 34 for receiving the screw 40. Other structure is possible. The apertures 26 and 34 can be larger in diameter than the cross-sectional diameter of the rod 38 and screw 40, if desired, to permit movement of the first device connecting member 18 relative to the rod 30 as shown by arrow 46 in FIG. 3, as well as transverse movement to the extent of the size of the aperture 26. Similarly, the size of the aperture 34 can permit movement of the second device connecting member 22 relative to the screw 40, as shown by arrow 50 in FIG. 3, as well as transverse movement to the extent of the size of the aperture 34. Also, the first device connecting member 18 can rotate about the rod 30, as shown by arrow 54, and second device connecting member can rotate about the screw 40, as shown by arrow 58.

The first device connecting member 18 and second device connecting member 22 are rotationally engaged to one another such that the first device connecting member 18 can rotate relative to the second device connecting member 22 as indicated by arrow 62 in FIG. 3. Any suitable connecting structure which will secure the first connecting member 18 to the second connecting member 22 and permit this rotation can be used.

The screw 40 can be any suitable spinal implant or pedicle screw or bolt. Threads 64 are provided for engaging the bone, however, other constructions for securing the device to bone are possible. The elongated shaft 70 can be of sufficient length that the second device connecting member 22 does not become disengaged. The shape of the screw head shaft may be varied to produce a desired motion path similar to a particular facet joint. For example, the screw shaft may have a curved shape. Alternatively, it is possible to place a head or cap unit on the screw 40. The head unit 80 (phantom lines in FIG. 1) would be an enlarged portion which could either be detachable from the screw 40 or form a permanent part thereof. The head 80 has a diameter larger than that of the aperture 34 such that the second device connecting member 22 cannot be removed from the screw 40. Other structure is possible.

The screw can also have an irregular cross section, such as an elipse, so that a connecting device can be attached which makes for the irregular shape and prevents rotation of the connecting device relative to the screw. The screw can alternatively have a protrusion or other engagement structure which engages a corresponding recession or cooperating engagement structure in the connecting device to prevent rotation.

It is also possible to limit the range of movement of the rod 30 within the first device connecting member 18. This can be accomplished by a blocking portion 82 that is provided on the rod 30 and is large enough so as not to permit passage through the aperture 26 of the first device connecting member 18. A second blocking member 84 can be provided on a portion of the rod 30 on the other side of the first device connecting member 22. The blocking members 82 and 84 can be fixed to the rod 30, or can be slidably engaged to the rod 30 and secured by suitable structure such as a set screw. Each of these blocking devices could provide progressive resistance (proportional to distance) with or without elastic properties. The blocking members 82 and 84 can be formed from a rigid material, or from an elastic material which will mimic the action of the ligaments. The elastic material can be such that a force is applied by the elastic material which is proportional to the distance traveled. Other structure is possible. Varying these parameters allows for closer reproduction of the ligament functions. The blocking devices can also prevent removal of the rod from the connectors. The blocking devices could also be connected to other parts of the construct, thus preventing any undesirable movement of the screw with respect to the vertebral body. For example, this could ensure a screw does not back out of the vertebral body. Such blocking devices could also be integral into the connector itself with the use of set screws, channels, and the like.

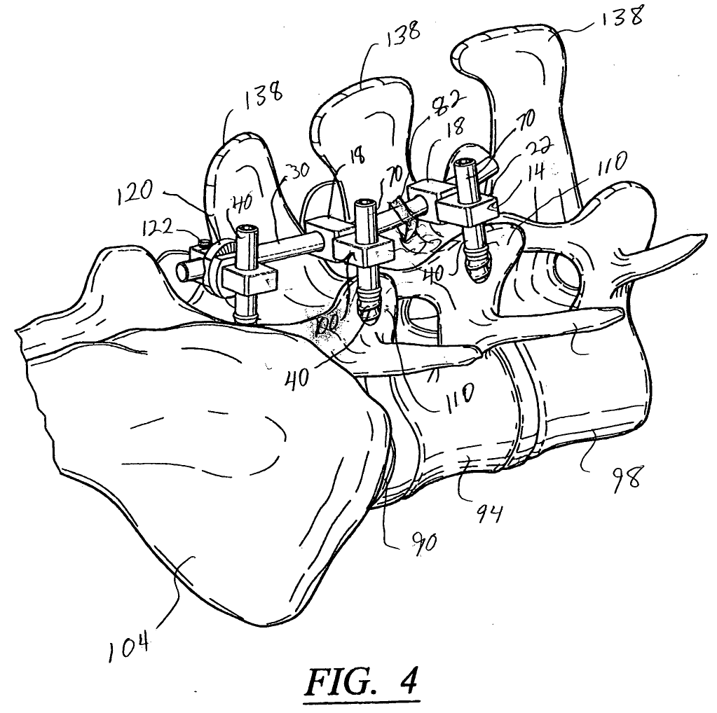

An installation of an artificial facet joint according to the invention is shown in FIG. 4. The invention can be utilized with any vertebra; however, there is shown the lumbar vertebrae 90, 94, and 98 adjacent to sacrum 104. The rod 30 is slidably engaged to the first connecting member 18 of the connector 14. The second device connecting member 22 is slidably engaged to the elongated shaft 70 of the pedicle screw 40. The apertures 26 and 34 can be coated with a friction reducing coating. The pedicle screw 40 is secured to the pedicle 110 of the vertebra 94. The screw 40 can be secured in the plane of the existing or former facet so as to better mimic the natural facet. Another connector 100 having a first device connecting member 18 and a second device connecting member 22, is connected to the pedicle 110 of the adjacent vertebra 90 by another screw 40. The provision of the connectors 14 and 100 on adjacent vertebrae with the rod 30 extending between them creates an artificial facet joint in which limited movement is permitted by the freedom of movement of the pieces of the joint, but which will not permit excessive movement. The action of the artificial facet also mimics the action of the ligaments which surround the spine to limit flexion of the spine.

The rod 30 is secured against excessive movement relative to the connectors 14 and 100 by clamping the rod 30 at some location. Any suitable structure for clamping the rod against movement is possible. There is shown in FIG. 4 a variable angle connector 120 which can be utilized. Such a connector is described in Simonson, U.S. Pat. No. 5,885,285, the disclosure of which is hereby incorporated fully by reference, however, any other suitable clamping or connection device can be utilized. The variable angle connector 120 can be secured to the spine by suitable structure such as another pedicle screw 40. The variable angle connector 120 has a set screw 122 which engages the rod 30 and prevents the rod 30 from moving relative to the variable angle connector 120.

There is shown in FIG. 4 two artificial facet joints. The connectors 14 and 100 with the rod 30 forms one joint. It is also possible to provide an artificial facet joint in which a connector 14 is provided on one adjacent vertebrae, and structure for securing the rod against axial movement relative to the spine is provided on the other adjacent vertebrae. This artificial facet joint would be formed by the connector 100 and structure for securing such as variable angle connector 120, but could be without any other connector such as connector 14. The rod 30 is thereby fixed on one side of the joint, and can slide through the connector 100 on the other side of the joint.

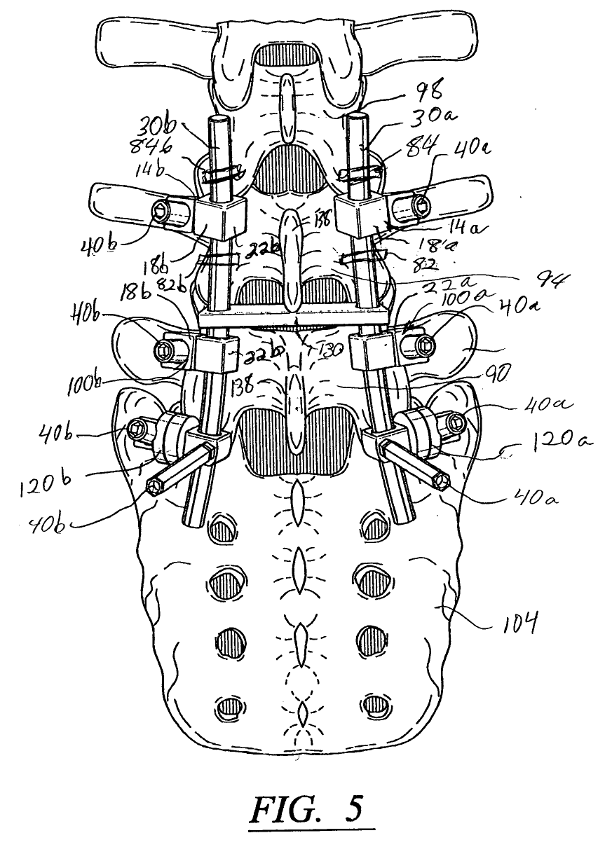

An artificial facet joint is created on each lateral side of the spine, as shown in FIG. 5. There is shown another assembly with spinal rod 30b slidably engaged to connectors 14b and 100b, which are comprised of first device connecting member 18b and second device connecting member 22b, and are also slidably engaged to screws 40b. Variable angle connector 120b or other suitable structure is utilized to secure the rod 30B in position.

A transverse member 130 is engaged to rods 30a and 30b. The transverse member 130 can have apertures which slidably engage the rods 30a and 30b. Other connection means are possible. The transverse connecter may connected to the screws 40a and 40b themselves to avoid rotation of the bone screws. The transverse member 130 can be in the form of a plate as shown or in any other suitable shape. The transverse member 130 provides torsional stability between the lateral sides of the artificial facet joint. The transverse member can be located between the spinous process 138 of the adjacent vertebrae 90 and 94.

The invention is made of suitable material such as surgical grade stainless steel. Any bio-compatible material with suitable strength can be utilized. The tolerances of the artificial facet joint can be created by variously sizing the rod 30, the screws 40, and the relative size of the apertures. Similarly, the transverse member 130 can be provided with apertures which permit a certain amount of movement. The amount of movement that will be appropriate will depend on the patient, the condition that is being treated, and the location in the spine where the artificial facet joint is located. Some portions of the spine are optimally more flexible than others. The connecting members could be differently dimensioned to provide different strength/flexibility characteristics.

This invention can be embodied in other forms without departing from the spirit or essential attributes thereof and, accordingly, reference should be had to the following claims rather than the foregoing specification as indicating the scope of the invention.

1. An artificial facet joint, comprising:
a pair of connectors, each connector comprising a first device connecting member having structure for sliding engagement of a rod, a second device connecting member having structure for sliding engagement of a screw, said first device connecting member and said second device connecting member being rotatably engaged to one another;
a spinal implant rod;
a pair of spinal implant screws; and
said first device connecting member of each of said connectors being slidably engaged to said rod, and said second device connecting member of each of said connectors being slidably engaged to a respective one of said pair of spinal implant screws, whereby said screws can be engaged to the pedicles on one lateral side of adjacent vertebrae and said rod and said connectors will limit movement of the joint; and,
structure for securing said spinal implant rod against axial movement relative to said spine.

2. The artificial facet joint of claim 1, wherein said structure for slidably engaging a rod is an aperture, and said structure for slidably engaging a screw is an aperture.

3. The artificial facet joint of claim 2, further comprising a second pair of connectors, a second spinal implant rod, and a second pair of spinal implant screws, whereby said second pair of screws can be engaged to the pedicles of the other lateral side of said adjacent vertebrae and said second rod and said second pair of connectors will limit movement of the joint on the other lateral side of said adjacent vertebrae.

4. The artificial facet joint of claim 3, further comprising a transverse member connected between said first and second rods.

5. The artificial facet joint of claim 4, wherein said transverse member is slidably engaged to said first and second rods.

6. The artificial facet joint of claim 5, wherein said apertures of said first device connecting member and said second device connecting member comprise a reduced friction coating.

7. The artificial facet joint of claim 1, further comprising structure for securing said rod to a portion of a spine.

8. The artificial facet joint of claim 7, wherein said structure for securing said rod comprises a clamp for said rod and structure for securing said clamp to a screw.

9. The artificial facet joint of claim 1, wherein said spinal implant rod comprises structure for engaging said first device connecting member so as to limit the sliding movement of the rod relative to the first device connecting member.

10. The connector assembly of claim 1, wherein said spinal implant screw comprises structure for engaging said second device connecting member so as to limit the sliding movement of the rod relative to the second device connecting member.

11. A connector for an artificial facet joint, comprising:
a first device connecting member having structure for sliding engagement of a spinal implant rod;
a second device connecting member having structure for sliding engagement of a spinal implant screw;
said first device connecting member and said second device connecting member being rotatably engaged to one another.

12. The connector of claim 11, wherein said structure for engaging said first device connecting member is an aperture, and said structure for engaging said second device connecting member is an aperture.

13. The connector of claim 12, wherein said apertures of said first device connecting member and said second device connecting member comprise a reduced friction coating.

14. A connector assembly for an artificial facet joint, comprising:
a connection device having a first connecting portion with structure for sliding engagement of a rod, and a second connecting portion with structure for sliding engagement of a screw;
a spinal implant rod slidably engaged to said first connecting portion; and,
a spinal implant screw slidably engaged to said second connecting portion.

15. The connector assembly of claim 14, wherein said structure for engaging a rod is an aperture.

16. The connector assembly of claim 14, wherein said structure for engaging a screw is an aperture.

17. The connector assembly of claim 14, wherein said spinal implant rod comprises structure for engaging said first connecting portion so as to limit the sliding movement of the rod relative to the first connecting portion.

18. The connector assembly of claim 14, wherein said spinal implant screw comprises structure for engaging said second connecting portion so as to limit the sliding movement of the rod relative to the second connecting portion.

19. An artificial facet joint, comprising:
a spinal implant rod;
a connector, said connector comprising a first device connecting member having structure for sliding engagement of said rod, a second device connecting member having structure for sliding engagement of a screw, said first device connecting member and said
second device connecting member being rotatably engaged to one another;
structure for securing said spinal implant rod against axial movement relative to said spine.

20. A method for creating an artificial facet joint, comprising the steps of:
providing a first pair of connectors, each connector comprising a rod connecting member having an aperture for engaging a rod, a screw connecting member having an aperture for engaging a screw, said rod connecting member and said screw connecting member being rotatably engaged to one another;
securing a first screw to a pedicle of a first vertebrae;
securing a second screw to a pedicle of a second vertebrae;
slidably engaging said screw connecting member of said first connector to said first screw, and said screw connecting member of said second connector to said second screw;
slidably engaging a spinal implant rod to said rod connecting member of said first connector and to said rod connecting member of said second connector; and
securing said rod.

21. The method of claim 20, further comprising the steps of:
providing a second pair of connectors, each connector comprising:
a rod connecting member having an aperture for engaging a rod;
a screw connecting member having an aperture for engaging a screw;
said rod connecting member and said screw connecting member being rotatably engaged to one another;
securing a first screw to a pedicle on an opposite lateral side of said first vertebrae;
securing a second screw to a pedicle on an opposite side of said second vertebrae;
slidably engaging said screw connecting member of said first connector of said second pair of connectors to said first screw, and said screw connecting member of said second connector of said second pair of connectors to said second screw;
slidably engaging a second spinal implant rod to said rod connecting member of said first connector and to said rod connecting member of said second connector on said opposite lateral side of said vertebrae; and
securing said second rod.

22. The method of claim 21, further comprising the step of attaching a transverse member between said spinal implant rods.

23. The method of claim 21, wherein said pedicle screws are positioned in the plane of the facet.

24. A spinal joint assembly comprising a spinal joint device joined to a spinal implant rod and capable of post-operative sliding movement relative to said rod.

25. The spinal joint assembly of claim 24, comprising structure for limiting the length of movement between said spinal implant rod and said spinal joint device.

26. A method of connecting a spinal joint assembly to a spine, comprising the steps of:
connecting a spinal implant rod to a spine, and
attaching a spinal implant device to said rod, said device being capable of post-operative sliding movement relative to said rod.

27. A spinal joint assembly comprising a spinal joint device joined to a spinal implant screw, said spinal joint device being capable of post-operative sliding movement relative to said screw.

28. The spinal joint assembly of claim 27, comprising structure for limiting the length of sliding movement between said spinal implant screw and said spinal joint device.

29. A method of connecting a spinal joint assembly to a spine, comprising the steps of connecting a spinal implant screw having a long axis to a spine, and connecting spinal implant device to said screw, said spinal implant device being capable of post-operative sliding movement along said long axis of said screw.

30. A bone implant screw for securing connected implants to the spine, said bone implant screw upon installation in the spine permitting dorsal movement relative to itself and said connected implants.

31. The bone implant screw of claim 30, wherein said screw comprises a post.

32. The bone implant screw of claim 30, wherein said movement further comprises rotation of said connected implants about an axis of said screw.

33. The bone implant screw of claim 30, wherein said screw comprises structure for limiting dorsal movement of said connected implants beyond a range of movement.
